SEQUENCE LISTING (4 sequences)

SEQ ID NO: 1 - DNA, 97 bp, Artificial Sequence
  Arbitrary Sequence
aaaaaaaaaaaaaaaaaaaaaattcttcttcttcttcttcttcttcttcttcttcttcttcttcttcttcttcttcttcttcttcttcttcttcttc

SEQ ID NO: 2 - DNA, 32 bp, Artificial Sequence
  Arbitrary Sequence
cttttccttctcaaaaaaaaaaaaaaaaaaaa

SEQ ID NO: 3 - DNA, 23 bp, Artificial Sequence
  Arbitrary Sequence
ttttttttttgagaaggaaaaag

SEQ ID NO: 4 - DNA, 23 bp, Artificial Sequence
  Arbitrary Sequence
ttttttttttagagaaagggaaa

(19) United States
(12) Patent Application Publication
(10) Pub. No.: US 2005/0214764 A1
(43) Pub. Date: Sep. 29, 2005

(54) Method for identifying, quantifying and/or characterizing an analyte

(76) Inventors: Emil Palecek, Cervinkova (CZ); Hans Kosak, Bonn (DE)

Correspondence Address: FISH & RICHARDSON P.C., PO BOX 1022, MINNEAPOLIS, MN, 55440-1022, US

(21) Appl. No.: 10/506,759

(22) PCT Filed: Mar. 4, 2003
(86) PCT No.: PCT/EP03/02202

§ 371 Date: May 4, 2005

(30) Foreign Application Priority Data
Mar. 8, 2002 (DE) 102 10 100.0
Jun. 17, 2002 (DE) 102 27 042.2

(51) Int. Cl. C12Q 1/68

(52) U.S. Cl. 435/6

(57) ABSTRACT

The invention relates to a method for identifying, quantifying and/or characterizing an analyte (10) contained in a first liquid. Said method is characterized by the following steps: a) bringing the analyte (10) into contact with a first probe (20) and with a second probe (22), each probe having an affinity to the analyte (20), and incubating the analyte. The affinity of the first probe (20) is effected by a specific affinity to at least one first binding site (12) of the analyte (20), and the incubating ensues under conditions under which the first probe (20), and the second probe (22) bind to the analyte (10). Other steps include: b) marking the first probe (20) with at least one first marker (24) that can be identified in an electrochemically specific manner at least when the probe (22) cannot already be identified in an electrochemically specific manner; c) marking the second probe (22) with at least one second marker (26) that can be identified in an electrochemically specific manner at least when this probe cannot already be identified in an electrochemically specific manner; d) separating the first probe (20) and second probe (22) that are bound to the analyte (10); e) detecting a first electrochemical signal Si1, which is caused by the separated first probe (20) or by the first marker (24) and detecting a second electrochemical signal Si2 caused by the separated second probe (22) or by the second marker (26), and; f) identifying, quantifying and/or characterizing the analyte (10) by using a ratio between the first Si1 and the second signal Si2.

The invention relates to a method for the proof, quantification and/or characterization of an analyte in a fluid.

From Sawada, K. et al. (2000), Analytical Biochemistry 286, pages 59 to 66, a method is known for the proof of a triplet expansion. The method is based on the proof of the triplet expansion in the patient genome through hybridization with a digoxygenin-labeled probe. The probe is used for a Southern hybridization. Bound probe is detected by binding an anti-digoxygenin-Fab-fragment labeled with alkaline phosphatase and then proving the alkaline phosphatase via a chemiluminescence reaction. The number of triplet repeats is estimated from the intensity of the thereby generated signal based on a linear ratio between the intensity and the number of triplet repeats. The disadvantage of this method is that it is not particularly sensitive and is relatively susceptible to faults due to the many steps of the method—1. Binding of the probe, 2. Binding of the antibody, 3. Prove of the antibody via enzyme reaction.

A further method for the proof of an expanded nucleotide repeat in genomic DNA is known from U.S. Pat. No. 5,695,933 A. With this, isolated genomic DNA is brought into contact with oligonucleotides which are ligatable with each other and which are complementary to the nucleotide repeat so that a hybridization takes place. The hybridized oligonucleotides are ligated with each other so that a multimer of the hybridized oligonucleotides is created. The genomic DNA is separated from the therewith hybridized multimer via denaturation. Then the hybridization and the ligation are repeated until a sufficient amount of multimers has been generated for a detection. Different oligonucleotides can also be used with this method. A disadvantage is that the expanded nucleotide repeats can only be proven by the length of the created multimers. This requires a gel-electrophoretic analysis of the multimers. Due to this the method is relatively costly and time-consuming.

Electrochemical methods can be used to examine and specify redoxactive analytes. Conversion of the analytes takes place on a working electrode. A reference electrode is used for the currentless measurement of the voltage. The current flowing through the working electrode or the voltage which drops between the working and the reference electrode is controlled via a counter electrode. The electro-chemical detection of analytes can be performed via potentiometry or amperometry. In a potentiometric measuring protocol the voltage dropping at the working and reference electrode is measured dependent on time. During this measurement a controlled current flow can be applied. In case of a constant current this measurement is called constant current chronopotentiometry. The examination of analytes adsorbed or complexed on a working electrode by means of constant current chronopotentiometry is also called constant current potentiometric stripping analysis (CCPSA). The cathodic constant current potentiometric stripping analysis includes a method in which analytes are oxidized successively by applying a positive current. Deductions concerning oxidizable analytes can be drawn from these voltage-dependent oxidation reactions. The anodic constant current potentiometric stripping analysis includes a method during which a negative current is applied so that deductions can be drawn concerning reducible analytes from the obtained voltage flow.

In an amperometric measuring protocol, the voltage dropping between the reference and the working electrode is changed via a counter electrode in accordance with a specified protocol. At the same time the current flowing over the working electrode is measured. Depending on the voltage which is applied, redoxactive analytes can be reduced or oxidized. By evaluating the current flow, deductions can be drawn concerning the analytes. An electrochemical measuring method with a stationary working electrode in which method a current-voltage characteristic curve is recorded is also known as voltammetry and the related graphic representation as voltammogram. For very sensitive measurements of redox processes, special measuring protocols were developed during which capacitive processes affecting the measurement which take place in addition to the redox processes are largely suppressed. A very efficient measuring protocol is the differential pulse voltammetry (DPV).

From U.S. Pat. No. 6,100,045 a method is known for the proof of an analyte. With this the analyte is labeled with a redoxactive substance and bound to a solid phase. The solid phase can involve magnetic micro particles. The solid phase is installed in the vicinity of an electrode. By means of the redoxactive label the binding of the analyte to the solid phase is proven as amperometric signal via the electrode. The known method can primarily provide information on the presence of the analyte but hardly on its properties. Furthermore it is not particularly sensitive. Its performance requires a device which is constructed relatively complicated. From U.S. Pat. Nos. 5,871,918 A and 6,346,387 B1 hybridization of an analyte, for example a DNA, is known with a catcher probe bound to an electrode. After the analyte is brought into contact with a reporter probe the electro-chemical proof of the hybridization takes place. For this, the bases of a reporter probe are converted with transition metal complexes into electro-chemically provable markers after binding to the target DNA. During this bases of the target DNA are also converted and generate an electro-chemical signal during proof. This limits the proof sensitivity of this method.

From Marrazza, G. et al. (2000), Clinical Chemistry 46:1, pages 31 to 37, a method is known for the electrochemical proof of PCR products. During this, a catcher DNA immobilized on an electrode is brought into contact with PCR-amplified DNA under hybridization conditions. The hybridization reaction on the surface of the electrode is followed by chronopotentiometric stripping analysis using daunomycin as indicator. The disadvantage of this method is that it always requires a coupling of the catcher DNA to the electrode and this electrode is then only suitable for proving a certain analyte.

From Authier et al., (2001), Anal. Chem. 73, pages 4450 to 4456, the non-specific binding of a nucleic acid as target DNA to an inner surface of a vessel is known. The target DNA is hybridized with an oligonucleotide labeled with a gold particle as reporter probe. The reporter probe is electro-chemically proven using microstrip electrodes which may only be used once. This method has many disadvantages. The use of a vessel to bind the target DNA makes it impossible to concentrate the target DNA in a small volume. This restricts the proof sensitivity. Furthermore the non-specific binding of substances, in particular the report probe, to the inner surface of the vessel can lead to a strong background signal and an interaction with the electro-chemical detection.

From WO 93/20230, a polarographic electrochemical method for the proof of a DNA hybridization is known. With this method, an electrode is first brought into contact with a single-strand DNA and a polarographic signal is measured for this single-strand DNA. Afterwards an oligonucleotide probe is added under hybridization conditions, excess reagent is removed and a polarographic signal is determined. By comparing the two determined signals it can be established whether a hybridization has occurred. With this method only a few pieces of information on the properties of the examined DNA can be obtained.

Object of the invention is to remove the disadvantages in accordance with the state of the art. In particular, as fast and as easy to implement as possible a method with high sensitivity for the proof, quantification and/or characterization of an analyte is to be specified. Characterization can take place with regard to the length or size of the analyte for example.

This object is solved by the features of claims 1 and 2. Useful embodiments result from the features of claims 3 to 33.

According to the invention, a method for the proof, quantification and/or characterization of an analyte contained in a first fluid is provided consisting of the following steps:

a) Bringing into contact and incubating the analyte with one each first and second probe exhibiting an affinity to the analyte, wherein the affinity of the first probe is caused by a specific affinity to at least one first binding site of the analyte and incubating takes place under conditions under which the first and the second probe bind to the analyte,

b) Labeling the first probe with at least one electro-chemically, specifically provable first marker, at least when it is not already electro-chemically, specifically provable,

c) Labeling the second probe with at least one electro-chemically, specifically provable second marker, at least when it is not already electro-chemically, specifically provable,

d) Abstracting the first and second probe bound to the analyte,

e) Detection of a first electro-chemical signal caused by the abstracted first probe or the first marker and a second electro-chemical signal caused by the abstracted second probe or the second marker and

f) Proof, quantification and/or characterization of the analyte by means of a ratio between the first and the second signal.

Furthermore a method for the proof, quantification and/or characterization of an analyte contained in a first fluid is provided consisting of the following steps:

a) Bringing into contact and incubating the analyte with one each first probe exhibiting an affinity to the analyte, wherein the affinity of the first probe is caused by a specific affinity to at least one first binding site of the analyte and incubating takes place under conditions under which the first probe binds to the analyte,

b) Labeling the first probe with at least one electro-chemically, specifically provable first marker, at least when it is not already electro-chemically, specifically provable,

c) Labeling the analyte with at least one electro-chemically, specifically provable second marker, at least when the analyte is not already electro-chemically, specifically provable,

d) Abstracting the analyte bound by the first probe and the first probe bound by the analyte,

e) Detection of a first electro-chemical signal caused by the abstracted first probe or the first marker and a second electro-chemical signal caused by the abstracted analyte or the second marker and

f) Proof, quantification and/or characterization of the analyte by means of a ratio between the first and the second signal.

The term “analyte” particularly applies to single or double-strand nucleic acids, proteins and other biomolecules. With the first or second probe, this can be monoclonal or polyclonal antibodies, antibody fragments, receptors, nucleic acids or ligands. In the sense of this invention the term “nucleic acids” also means, in addition to DNA and RNA, nucleic acid analogues such as peptide nucleic acids (PNA) as well as hybrids and chimerics of DNA and RNA and analogues of nucleic acids.

Concerning electro-chemical provability, “specific” means that the first or second probe, the analyte or the first or second marker can each be proven electro-chemically, in other words by a redox reaction, separately from each other and if necessary from other substances for example additional substances contained in the first fluid. This may be possible with a specific signal or in that the signal can be differentiated in another way from other signals, in particular also background or interference signals. For example this is possible with a differential detachment of the first probe and/or cleaning processes.

For specific proof, the first and/or second probe and/or the analyte can each be labeled with at least one electro-chemically provable marker or an electro-chemically provably marker group. Significantly more intense signals can be generated by labeling with several markers or marker groups than with an electro-chemically self-provable analyte or an electro-chemically self-provable probe. The sensitivity and the specificity of the method can be increased by this. Also when the analyte or the probe are electro-chemically provable without labeling, such as for example nucleic acids which contain a purine base such as guanine or adenine, they can be labeled. Labeling can take place in that an electro-chemically provable marker binds via an affinity molecule such as for example avidin to an affinity molecule corresponding to it, such as for example biotin, located on the analyte or the first or the second probe. The term “affinity molecule” means a molecule which exhibits a specific high affinity to a corresponding additional affinity molecule. The labeling steps lit. b and lit. c must be executed before step lit. e but can be executed each before or after step lit. a or lit. d.

The binding of the second probe can take place specifically or non-specifically. With an analyte consisting of nucleic acid, a specific binding preferably means a sequence-specific hybridization. It can also be a sequence-specific binding of a protein. The binding of a first or second probe consisting of nucleic acid to an analyte consisting of nucleic acid can also take place via a Hoogsteen base pairing which forms a triplex structure. This happens preferably with a binding of PNA to an analyte consisting of double-strand DNA. A nucleic acid indicator such as a DNA intercalator which binds sequence-independently to the analyte can be used for example as the second probe.

The term “abstracting” means a largely separation of the bound from the unbound first or second probes or of the bound from the unbound analyte. For example this can take place by breakdown of unbound first or second probes or of the unbound analyte or by centrifuging, binding or washing. Separation of the bound from the unbound first probe and the analyte bound by the first probe from the unbound analyte can also be executed in one step, for example by centrifuging or by binding and then washing the formed complex consisting of analyte and first probe. With washing the complex is placed in an additional fluid. Detection of the first and the second signal can take place simultaneously or consecutively and with one and the same or different electro-chemical methods. Detection also includes a quantification of the signals if necessary. This can also include a mathematic operation, in particular an integration.

The essential feature of the method according to the invention is the generation of two electro-chemical signals which are characteristic of the analyte which are put into a ratio to each other. This ratio permits specific statements to be made about the analyte which would not be possible with only hybridization with only one probe generating a signal or would only be possible with additional effort such as for instance gel electrophoresis. The method is particularly well suited to characterizing a nucleic acid by its length. If the second signal is caused by a DNA intercalator as second probe for example and if the DNA has a specific binding site for a first probe, the ratio of the first signal to the second signal provides information on the length of the nucleic acid. Furthermore the length of nucleic acids with frequently repeated sequences can be determined for example in that the first probe binds specifically to a sequence which only occurs once in the molecule while the second probe binds specifically to the sequences which are repeated. The first signal can represent an internal standard for the second signal which standard, particularly when an expected first signal is known, permits a differentiation of a specific portion of a non-specific portion of the second signal. This achieves a high degree of sensitivity or specificity of the method. Non-specific second signals can for example be caused by incompletely abstracted, unbound second probes.

An electrode used for the detection is preferably not brought into contact with the first fluid. This can significantly increase the sensitivity of the method since substances contained in the first fluid such as for example blood serum can not thereby bind non-specifically to the electrode and generate non-specific signals during the detection. Non-specific background signals can thus be largely avoided. This can be achieved for example by separating the analyte from the first fluid before step lit. e and transferring it to a second fluid. Preferably the analyte is separated from the first fluid before the execution of step lit. a and transferred to a second fluid. Thereby substances contained in the first fluid can be separated which bind to the first or second probe, break it down or interfere with its function in other ways. Furthermore substances can be separated which interfere with the detection. These can be for example substances which generate a similar electro-chemical signal as the first or second probe. These can also be substances which interfere with the function of the electrodes used for the electro-chemical detection. For separation, the analyte can be bound, in particular specifically, by a catcher molecule.

The binding of the analyte to the catcher molecule can be performed via a, preferably sequence-specific, hybridization. Particularly before the analyte is bound, the catcher molecule can be immobilized on a first or second surface.

This simplifies the separation of the analyte from the first fluid. The analyte can be concentrated in a small volume because of the immobilization of the catcher molecules. This increases the proof sensitivity of the method.

The catcher molecule can be a nucleic acid, an analogue of a nucleic acid, in particular a peptide nucleic acid (PNA), an antibody or a receptor. Preferably the catcher molecule has an affinity molecule, in particular streptavidin, avidin or biotin, or a biotinylated oligonucleotide. This affinity molecule can be used either to immobilize the catcher molecule on the first or second surface or to bind the analyte.

In a preferred embodiment, the first or second probe or the analyte contains an affinity molecule, in particular biotin, avidin or streptavidin. The affinity molecules must be selected so that bindings cannot take place which prevent the performance of the method according to the invention. In particular bindings between the first and the second probe or bindings of the first probe to the analyte should not take place via the affinity molecules. This provides a simple way to separate the analyte from the first fluid. Furthermore the affinity molecule enables a binding of a marker to the first or second probe or to the analyte or also an immobilization of the analyte. The analyte can be a nucleic acid, in particular with a poly-T-end or a poly-A-end. The nucleic acid can be double or single-stranded. A sequence-specific binding is also possible on a double-stranded nucleic acid, for example via a Hoogsteen binding of a first or second probe consisting of PNA. The poly-T-end or the poly-A-end enables the binding of the analyte to a nucleic acid used as catcher molecule having a poly-A-sequence or a poly-T-sequence. This makes it simple to separate the analyte from the fluid. Furthermore a poly-T-end permits a labeling with several osmium complexes. In a special embodiment of the method the characterization takes place by determining the length of the nucleic acid.

Preferably the analyte is amplified before or during step lit. a by means of a nucleic acid amplification reaction, in particular a PCR. This significantly increases the sensitivity of the method. The product of the amplification reaction is then essentially proven, quantified and/or characterized as the analyte. This also applies when the product does not completely match the analyte due to the primers used for the amplification reaction or when a product of the amplification reaction is concerned which is complementary to the analyte. With the PCR, the catcher molecule or the first or second probe can be used as primer. Specific binding sequences can be introduced into the analyte by means of the PCR.

The analyte can be immobilized on the first or second surface before step lit. d, in particular before step lit. a. This can also be done via the binding to the catcher molecule. This simplifies the separation from the first fluid and the abstraction in accordance with lit. d which can then be performed for example via washing of the first surface. The first surface can be the surface of a particle, in particular a superparamagnetic one. A superparamagnetic particle can be easily separated by means of a magnetic field. The particle can also otherwise be separated by means of centrifugation. It enables the analyte to be concentrated. Furthermore the particle makes it possible to provide a large binding surface. The particle preferably has a diameter of 10 nm to 100 μm, in particular 1 to 10 μm. This particle size makes it possible to suspend the particles in the fluid and thus establish an intensive contact of the analyte with the first and, if appropriate, the second probe.

In a preferred embodiment, the analyte is immobilized on the first surface before performance of step lit. a via a catcher molecule. The catcher molecule can be immobilized on the first surface before or after the binding to the analyte. A washing step is then performed during which the first fluid is removed and is replaced by a second fluid. The second fluid can be selected such that optimal conditions are provided for the binding of the first or the first and the second probe during step lit. a. Step lit. d is preferably performed in that the immobilized analyte is washed with the first or first and second probe bound thereto to remove the unbound first or first and second probe. During this, the second fluid can be replaced by a third fluid. The third fluid is preferably selected so that the detection according to lit. e can be performed therein under optimal conditions.

Preferably the second surface is an electrode used for detection, in particular containing an electrically conductive plastic or an electrically conductive polymer, mercury, amalgam, gold, platinum, carbon or indium tin oxide.

Preferably the second probe exhibits a specific affinity to at least one specific second binding site of the analyte. From the ratio between the first and the second signal, information can then be obtained concerning the ratio of the number of the first binding sites to the number of the second binding sites. Preferably the analyte exhibits a known number of first binding sites and an unknown number of second binding sites. The characterization can then take place with the determination of the unknown number of the second binding sites by the ratio of bound first probe to the bound second probe or the ratio of the first signal to the second signal. The signal ratio or the number of the second binding sites determined from this can be correlated with the length of the analyte. The analyte can be a DNA fragment which exhibits repetitive sequences due to a triplet expansion disease such as the fragile X syndrome, Huntington's disease, bulbar muscular atrophy, type I spinocerebral ataxia, myotonic dystrophy or Friedreich's ataxia. Such diseases are usually proven via a polymerase chain reaction with subsequent Southern blot. In relation to the method according to the invention with which the repetitive sequences can be used as second binding sites, this requires a very great amount of effort and time.

In a preferred embodiment, the first probe is released from the analyte and/or the analyte is released from the first or second surface between step lit. d and step lit. e, in particular via heat denaturation, chemical denaturation, enzymatic digestion or chemical breakdown. Release from the surface makes it possible to concentrate the analyte or the first probe in a very small volume of fluid and thus increases the sensitivity of the method. In addition it enables close contact of the first probe or the analyte with the electrode used for detection. Furthermore it is thus possible to execute the detection without the electrode coming in contact with the first surface. This prevents possible disturbances of the electro-chemical detection by the first surface. Materials can also be used as the first surface which would normally disturb the electro-chemical detection. For example streptavidin-coated particles can be used as the first surface and cysteine-labeled PNA can be used as the first probe without disturbances being able to occur during the electro-chemical detection of the cysteine-labeling by the cysteine residues of streptavidin. Furthermore the release of the first probe from the analyte makes it possible to detect the first and the second signal separately from each other. This means that the method can also be performed when the first and the second signal are identical, because for example it is caused by identical markers.

It is also possible to release the first and the second probe from the analyte or the first probe from the analyte and the analyte from the first or second surface separately from each other and then to perform detection. A differentiation between identical signals by the molecule which caused them is thus also possible. Furthermore concentration in a small volume of fluid is possible.

Alternately it is also possible to release the first marker and/or the second marker, in particular separate from each other, from the first and/or second probe or the analyte and then to perform detection. Release can be performed via enzymatic digestion or chemical breakdown. For example the first and/or the second marker could be bound each via a nucleic acid sequence with a specific restriction cleavage site to the first or second probe or the analyte. Specific release is then possible via restriction enzymes.

The first and/or second probe can be a nucleic acid or an analogue of a nucleic acid, in particular a peptide nucleic acid (PNA). Preferably the first and/or second probe binds sequence-specifically to the analyte, particularly via hybridization. For example the probe can be a nucleic acid which is complementary to a sequence of the analyte. The probe can also be an antibody which recognizes a certain amino acid sequence in an analyte consisting of a protein.

The first or second signal can each be caused by a catalytic hydrogen release caused by the first or second marker. Preferably the first and/or the second marker can be reversibly reduced or oxidized. The first or the second marker can have an osmium complex, a nano gold particle, a cysteine, ferrocenyle, daunomycin, benzoquinone, naphthoquinone, anthraquinone or p-aminophenol group or a dye, in particular indophenol, thiazine or phenazine.

Preferably the first or the second probe is labeled by several markers. This makes it possible to use a probe for different analytes which are electro-chemically differently detectable. Preferably the first or second probe has a linear primary structure on whose one end the marker is located. Since the marker is located on the end, the danger of affecting the interaction between the first or the second probe with the analyte is minimized.

Detection of the first and the second signal can take place on the same electrode and/or via the same electro-chemical method of proof. Detection is preferably performed by means of cathodic stripping voltammetry (CSV), squarewave voltammetry, cyclic voltammetry or chronopotentiometry. Detection can take place by means of a reversible redox process or a catalytic hydrogen development.

Examples will now be used to describe the invention in more detail. The figures are listed below:

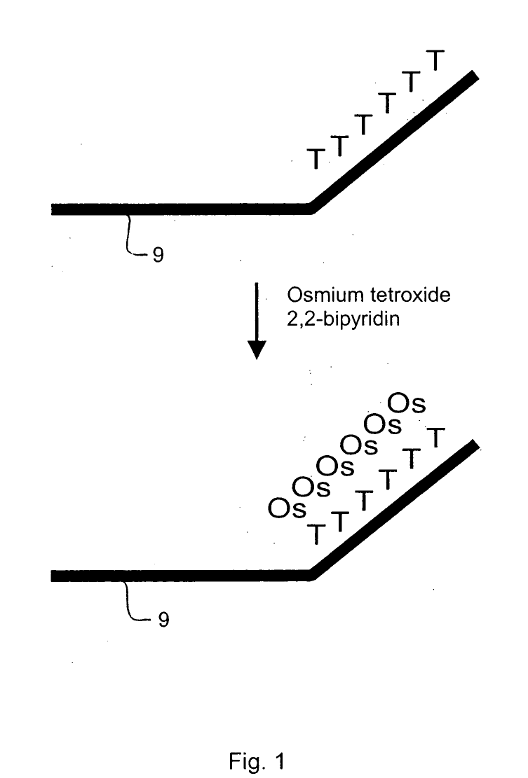

FIG. 1 A schematic presentation of the labeling of a first probe by osmium tetroxide and 2,2′-bipyridin,

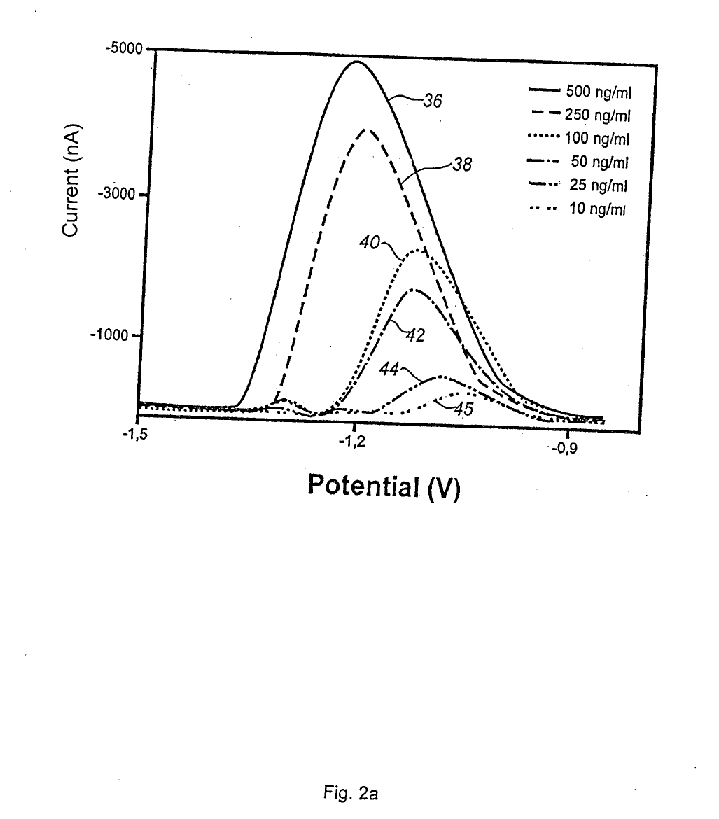

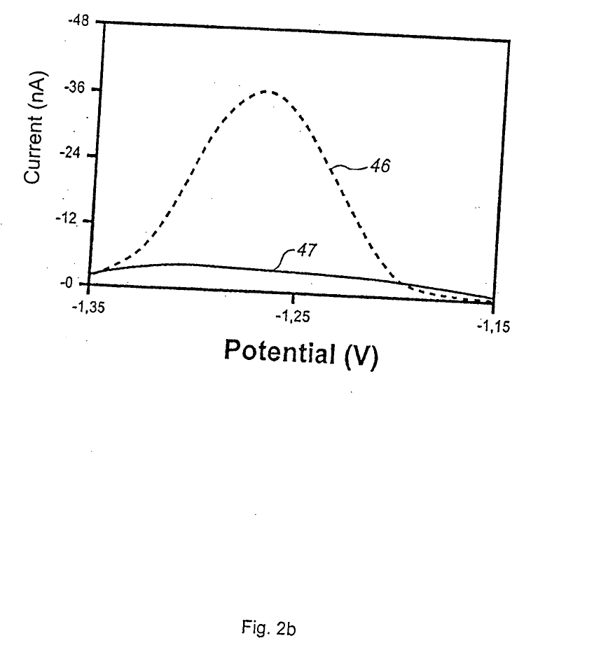

FIG. 2a, b DPV voltammograms for determination of the detection limit,

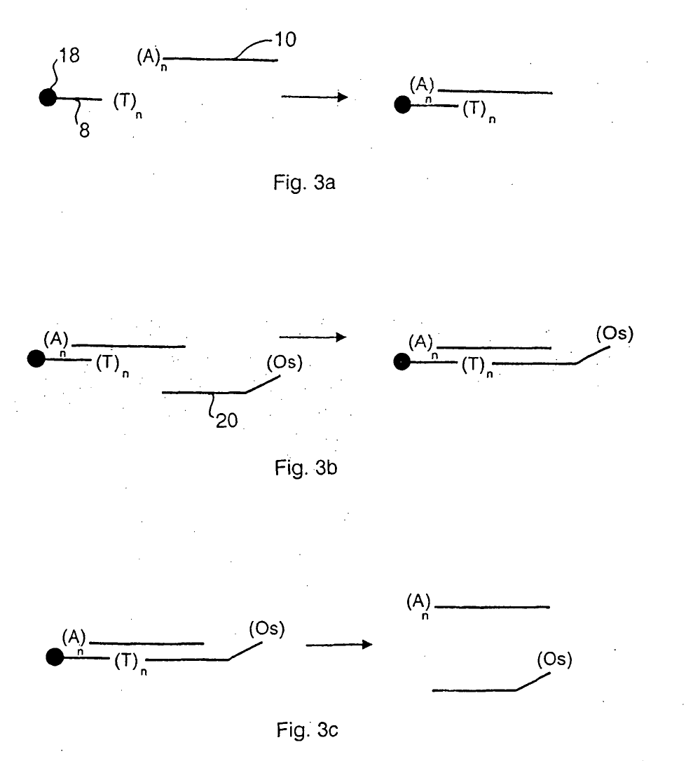

FIG. 3a, b, c Schematic presentation of the proof of an analyte consisting of nucleic acid via hybridization with an osmium-labeled first probe,

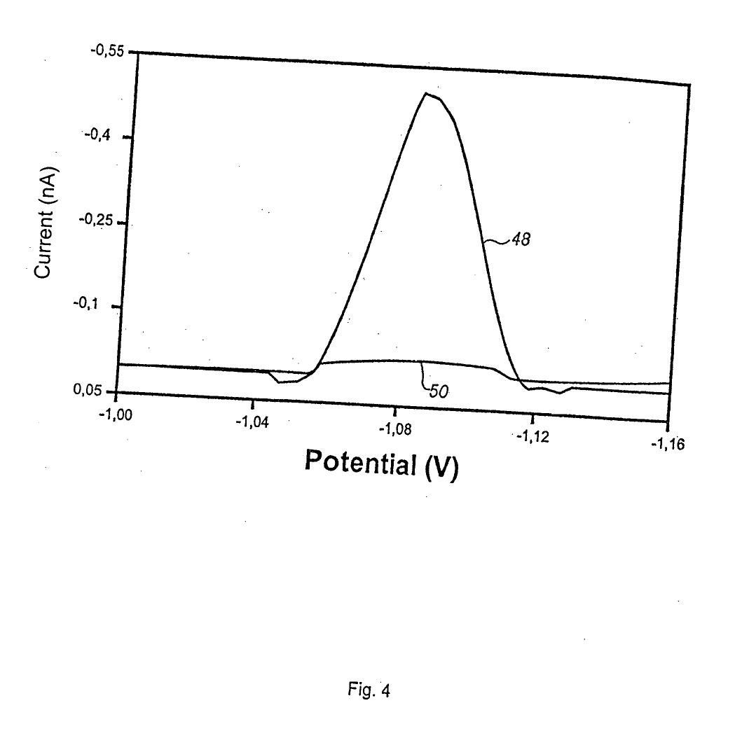

FIG. 4 DPV voltammogram for the proof of the anylyte consisting of nucleic acid via hybridization with an osmium-labeled first probe,

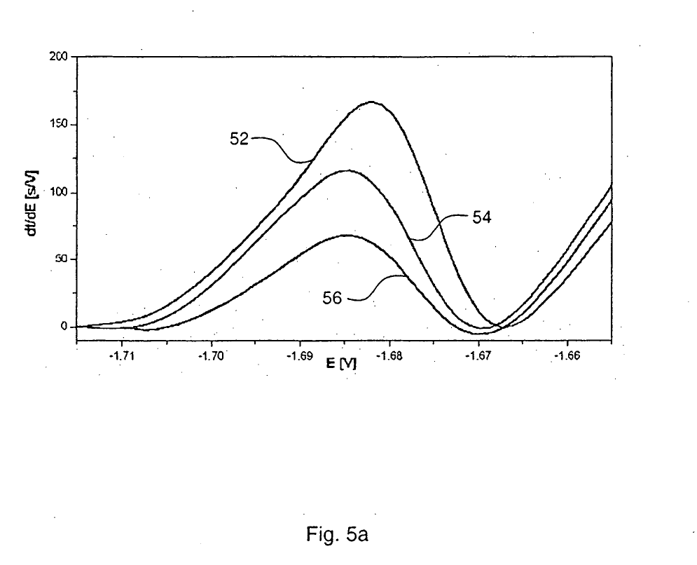

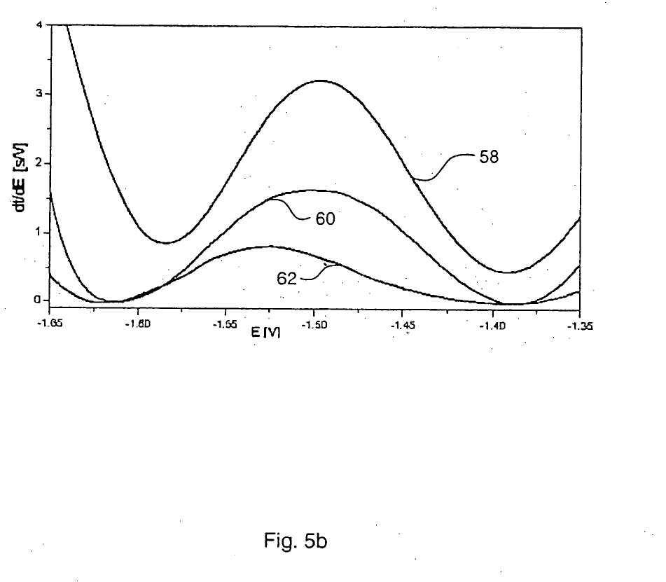

FIG. 5a, b Voltammograms of a chronopotentiometric stripping analysis (CPSA) for the determination of sensitivity of cysteine PNA probes,

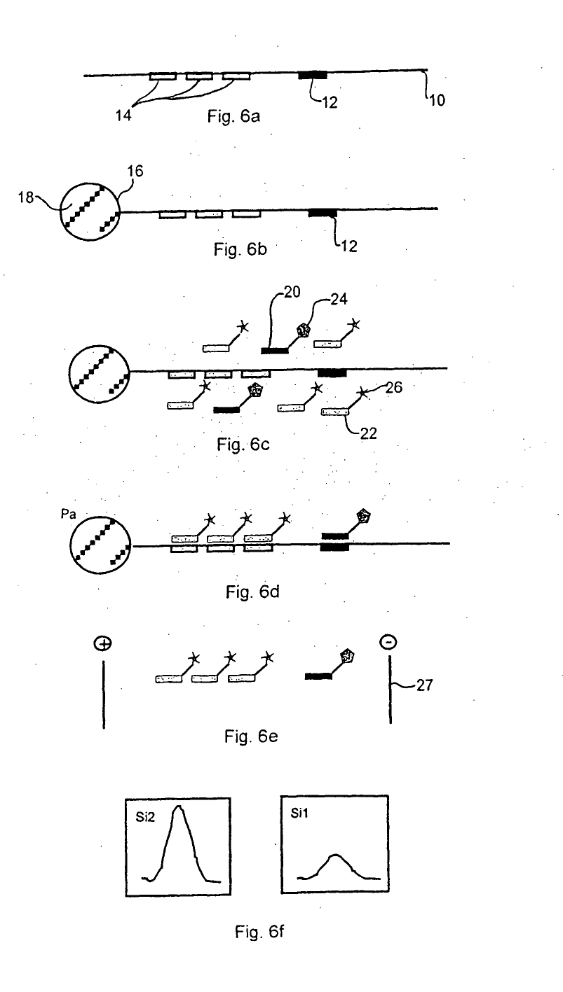

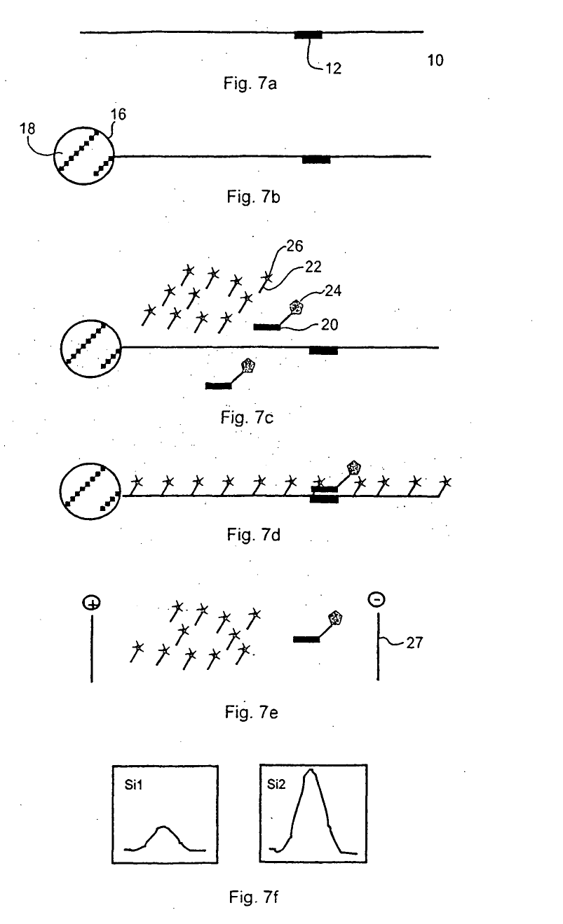

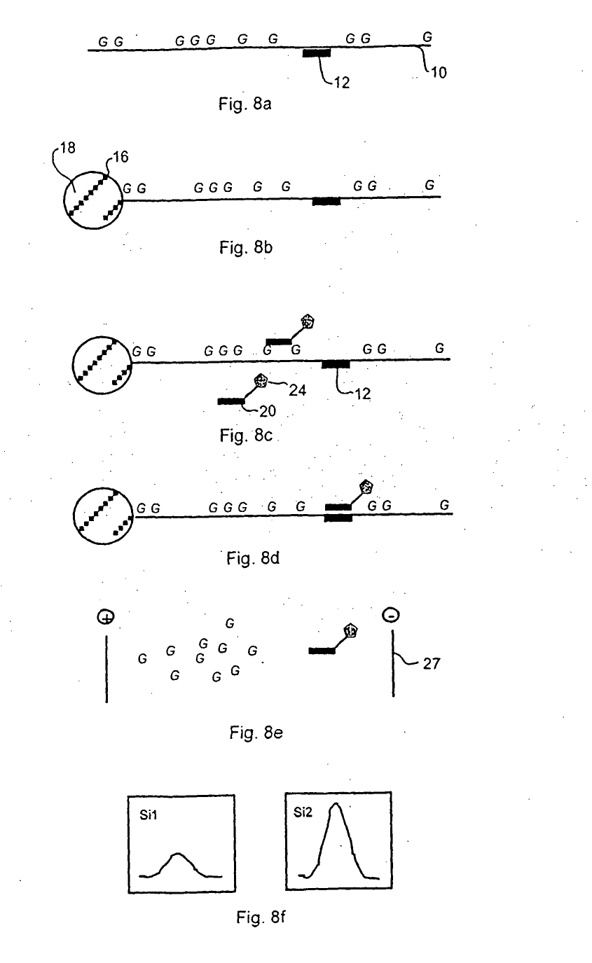

FIG. 6a-f Schematic presentation of a method according to the invention with a first and a second probe,

FIG. 7a-f Schematic presentation of a method according to the invention with a sequence-specific first and a sequence-non-specific second probe,

FIG. 8a-f Schematic presentation of a method according to the invention, wherein a first probe and the analyte are used to generate electro-chemical signals and

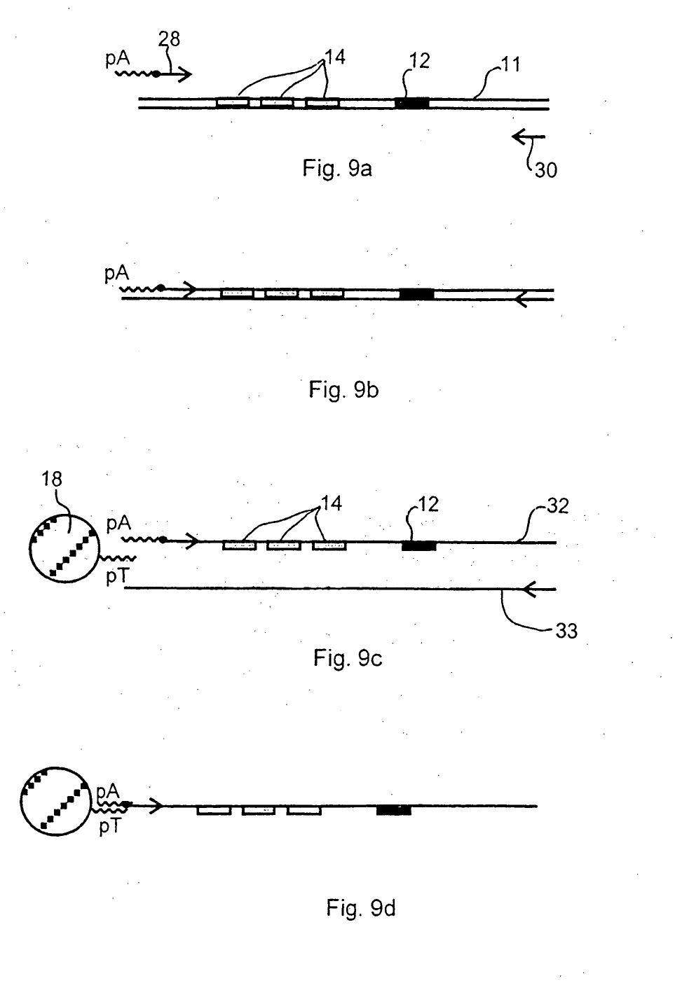

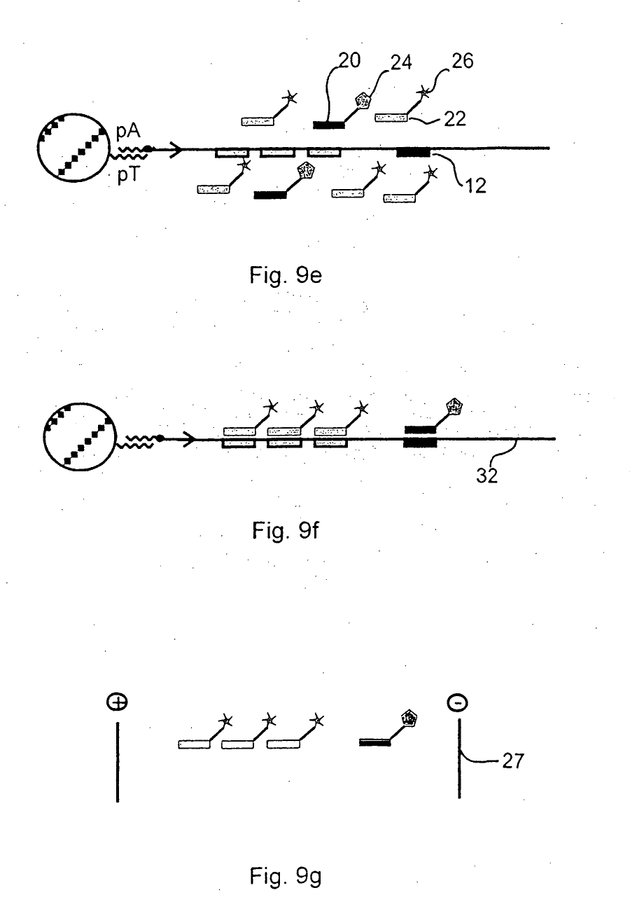

FIG. 9a-g A schematic presentation of the determination of the number of reversible sequences specific for a triplet disease.

To produce an osmium-labeled probe, a 97mer with the sequence 5-AAA AAA AAA AAA AAA AAA AAA ATT CTT CTT CTT CTT CTT CTT CTT CTT CTT CTT CTT CTT CTT CTT CTT CTT CTT CTT CTT CTT CTT CTT CTT CTT C-3 (SEQ ID NO: 1) from the enclosed sequence listing was incubated for 180 minutes at 37° C. with 2 mmol/l OSO₄, 2 mmol/l 2,2′-bipyridin in 100 mmol/l Tris-HCl, pH 7.0. Free OSO₄ and bipyridin was then removed by dialysis against deionized water for 5 hours. The labeling is shown in FIG. 1 schematically. An oligonucleotide with a first sequence 9 complementary to an area of an analyte consisting of nucleic acid and a second sequence containing many thymine residues T is treated with osmium tetroxide and 2,2′-bipyridin. The thymine residues form specific osmium containing complexes Os which can be proven as electro-chemical markers.

To determine the detection limit of the probe made like this, 3 μl each of a 10, 25, 50, 100, 250 and 500 ng/ml of the osmium-labeled probe in Britton Robinson buffer (0.04 mol/l H₃BO₃, 0.04 mol/l H₃PO₄ and 0.04 mol/l CH₃COOH mixed with 0.2 mol/l NaOH), pH 3.9 are measured by means of differential pulse voltammetry (DPV) with a scan rate of 10 mV/s, an amplitude of 50 mV/s, an absorption time of 240 s and a scan time of 120 s to 240 s. For the result, see the DPV voltammogram shown in FIG. 2a. The curves 36, 38, 40, 42, 44 and 45 correspond respectively to 500, 250, 100, 50, 25 or 10 ng/ml of the osmium-labeled probe. FIG. 2b shows a DPV voltammogram 46 generated with the same method with 3 μl of a solution containing 250 pg/ml osmium-labeled probe and a DPV voltammogram 47 generated without probe. The detection limit of the osmium-labeled probe is thus under 250 pg/ml. This corresponds to 25 attomol probe.

FIG. 3a shows an analyte 10 consisting of nucleic acid with a poly-A-strand (A)ₙ on one end which is brought into contact with a particle coated with poly-T (T)ₙ as catcher molecule 8. The analyte 10 binds to the particle 18 via hybridization of (A)ₙ with (T)ₙ. The particle-bound analyte is brought into contact with an osmium-labeled first probe 20 (FIG. 3b). The first probe 20 has a sequence which is complementary to the non hybridized area of analyte 10. Under the selected conditions, the first probe 20 binds to analyte 10. Unbound first probe 20 is removed by washing the particles. The bound first probe 20 is then released from analyte 10 by heat treatment (FIG. 3c). The first probe 20 can be electro-chemically detected by means of its osmium-label.

The proof can be performed with the following components, for example:

1. Analyte:

(SEQ ID NO: 2)

5′-CTT TT CCT TCT CAA AAA AAA AAA AAA AAA AAA

2. Probe I with complementary sequence to the analyte:

| | |
|---|---|
| 5′-TTT TTT TTT TGA GAA GGA AAA AG | (SEQ ID NO: 3) |

3. Probe II without complementary sequence to the analyte:

| | |
|---|---|
| 5′-TTT TTT TTT TAG AGA AAG GGA AA | (SEQ ID NO: 4) |

The thymine residues of the probes I and of the probe II used for control were labeled as described above with osmium tetroxide and 2,2′-bipyridin.

To immobilize the analyte, 40 μl of superparamagnetic particles on which oligo-T-25 mers of the company Dynal, Norway are immobilized are added to 40 μl of a solution containing 10 μg/ml of the analyte in 0.1 mol/l NaCl, 0.05 mol/l phosphate buffer, pH 7.4 (incubation buffer). The suspension is incubated for 30 minutes at room temperature. The particles are washed twice in 100 μl incubation buffer and placed in a volume of 40 μl.

To hybridize the analyte with the probe, 40 μl of the incubation buffer containing 400 ng/ml of the probe are added to the particles and incubated for 30 minutes at room temperature. Unbound probes are removed by four washings in 100 μl incubation buffer. The bound probe is released by heating to 85° C. and abstracted by separation of the supernatant containing the probe. For electro-chemical detection of the osmium-labeled probe, 3 μl of the supernatant are analyzed by DPV with an absorption time of 2 minutes in Britton-Robinson buffer, pH 3.87. To determine a non-specific binding, the analyte is brought into contact and incubated with probe II in a separate preparation under identical conditions.

The DPV voltammogram illustrated in FIG. 4 shows a clear signal 48 of probe I and a very weak signal 50 of probe II used for control.

To determine the sensitivity of the chronopotentiometric stripping analysis (CSPA) when using cysteine-PNA probes, various concentrations of cysteine-PNA (cys-PNA) in 5 mmol/l phosphate buffer, pH 7.0 were absorbed on an electrode for an absorption time of 7 minutes. FIG. 5a shows a DPV diagram then taken in 0.2 mol/l ammonium phosphate buffer, pH 8.5 at a stripping current of −2 μA with the hydrogen peak curves 52, 54 and 56 of 0.375, 0.180 and 0.094 ng/ml cys-PNA each. The detection limit of cys-PNA was under 0.09 ng/ml. This corresponds to 36 attomol cys-PNA in 1 μl. FIG. 5b shows a DPV diagram of Brdicka's signal curves 58, 60 and 62 of each 0.750, 0.375 and 0.187 ng/ml cys-PNA in 1 mmol/l Co³⁺, 0.1 mol/l ammonium phosphate buffer, pH 9.47. The detection limit of cys-PNA was under 0.19 ng/ml.

FIG. 6a-f show a schematic presentation of a method according to the invention during which a first probe 20 and a second probe 22 bind sequence-specifically to the analyte 10. FIG. 6a shows an analyte 10 with a singular first binding site 12 and three repetitive second binding sites 14. The analyte 10 is immobilized on a first surface 16 of a particle 18 (FIG. 6b). The analyte 10 is brought into contact with the first probe 20 and the second probe 22 (FIG. 6c). The first probe 20 has an affinity to the first binding site 12 and the second probe 22 has an affinity to the second binding sites 14. The first probe 20 is labeled with a first electro-chemical marker 24 and the second probe 22 is labeled with a second electro-chemical marker 26.

Under the selected conditions, the first probe 20 binds to first binding site 12 and the second probe 22 binds to second binding sites 14 (FIG. 6d). Unbound probes are removed by washing. The probes are released from the analyte and, for the proof, are brought into contact with a second surface 27 used as an electrode (FIG. 6e). The signal Si1 caused by marker 24 and the signal Si2 caused by the marker 26 are measured (FIG. 6f) and put into a ratio to each other. The ratio of Si2 to Si1 corresponds to the ratio of the number of second binding sites 14 to first binding site 12.

FIG. 7a-f show a schematic presentation of the method according to the invention, wherein a first probe 20 binds sequence-specifically to the analyte 10 and a second probe 22 binds sequence-non-specifically to the analyte 10.

FIG. 7a shows an analyte 10 which has a singular first binding site 12. The analyte 10 is immobilized on a first surface 16 of a particle 18 (FIG. 7b). The analyte 10 is brought into contact with the first probes 20 and the second probes 22 (FIG. 7c). The first probe 20 has a binding affinity to the first binding site 12. The second probe 22 has a sequence-non-specific affinity to analyte 10. The second probe 22 can for example be a DNA intercalator. The first probe 20 is labeled with a first electro-chemical marker 24 and the second probe 22 is labeled with a second electrochemical marker 26.

Under the selected conditions the first probe 20 and the second probe 22 binds to the analyte 10 (FIG. 7d). Unbound first 20 and second 22 probe are removed by washing. Bound first 20 and the second probe 22 are released from the analyte and are brought into contact with a second surface 27 used as a proving electrode (FIG. 7e).

The first signal Si1 caused by the marker 24 and the second signal Si2 caused by the marker 26 are measured (FIG. 7f) and put into a ratio to each other. The ratio corresponds to the ratio of the first binding site 12 to the total length of the analyte 10.

FIG. 8a shows an analyte 10 consisting of nucleic acid with a singular first binding site 12 and a certain number of guanine residues G. The analyte 10 is immobilized on a first surface 16 of a particle 18 (FIG. 8b). It is brought into contact with a first probe 20 which is labeled with an electrochemical marker 24 and has a specific affinity to the first binding site 12 (FIG. 8c). Under the selected conditions the first probe 20 binds to the first binding site 12 (FIG. 8d). Unbound first probe 20 is removed by washing. First probe 20 bound to the analyte 10 and the guanine residues G are released from or out of the analyte 10, for example by acidic hydrolysis, and are brought into contact with a second surface 27 used as proving electrode (FIG. 8e). The signal Si1 generated by G and the signal Si2 generated by the marker 24 are measured (FIG. 8f) and put into a ratio to each other. The ratio of the signals Si1 to Si2 corresponds to the ratio of numbers of the first binding site 12 to the total number of G in the analyte 10.

FIG. 9a shows a double-stranded DNA fragment 11 which has an unknown number of repetitive sequences specific for a triplet disease containing one second binding site 14 each and a singular sequence containing a first binding site 12. The DNA fragment is brought into contact with a first primer 28 having a poly-A-portion pA on its 5′ end and a second primer 30. The first 28 and second primers 30 each bind to a DNA strand of the DNA fragments 11 and thereby surround the second binding sites 14 and the first binding site 12 (FIG. 9b).

Via an amplification reaction an amplification product 32 is generated which contains the second binding sites 14 and the first binding site 12 as well as the poly-A-portion on one 5′ end (FIG. 9c). The amplification product 32 is separated from its counter strand 33 by denaturation and brought into contact with a superparamagnetic particle 18 having a poly-T-probe as catcher molecule pT. The poly-A-portion pA thereby binds specifically to the poly-T-probe pT (FIG. 9d).

The amplification product 32 bound to the particle 18 is brought into contact with a first probe 20 which has a specific affinity to the first binding site 12 and a second probe 22 which has a specific affinity to the second binding sites 14. The first probe 20 is labeled with an electro-chemical marker 24 and the second probe 22 is labeled with an electro-chemical marker 26 (FIG. 9e).

Under the selected conditions, the first probe 20 binds to the first binding site 12 and the second probe 22 binds to the second binding sites 14 (FIG. 9f). The excess of unbound first probe 20 and second probe 22 is removed by washing.

Bound first 20 and second probes 22 are released from the amplification product 32 and brought into contact with a proving electrode 27 (FIG. 9g). The electrochemical signals caused by the markers 24 and 26 are recorded separately from each other. The number of repetitive sequences is determined from the ratio of the signals generated by the markers 24 and 26. The method is thus suited to the proving and identifying of a triplet disease.

1-34. (canceled)

34. A method for characterizing an analyte (10) contained in a first fluid, comprising the steps of:
contacting the analyte (10) with a first probe (20) and a second probe (22) under conditions in which the first and second probe bind to the analyte, wherein the first probe binds specifically to at least one first binding site (12) of the analyte (10);
labeling the first and second probes with a first and a second electro-chemical label, respectively, if the first and second probes are not already electro-chemically reactive;
abstracting the first and second probes bound to the analyte;
detecting a first electro-chemical signal (Si1) caused by the first-labeled probe (20) and a second electro-chemical signal (Si2) caused by the second-labeled probe (22); and
determining the ratio between the first (Si1) and the second signal (Si2), thereby characterizing the analyte (10).

35. A method for characterizing an analyte (10) contained in a first fluid, comprising the steps of:
contacting an analyte (10) with a first probe (20) under conditions in which the first probe binds to the analyte, wherein the first probe binds specifically to at least one first binding site (12) of the analyte (10);
labeling the first probe and the analyte with a first electro-chemical label and an analyte electrochemical label, respectively, if the first probe and analyte are not already electro-chemically reactive;
abstracting the analyte bound by the first probe and the first probe bound to the analyte;
detecting a first electro-chemical signal (Si1) caused by the first-labeled probe (20) and an analyte electro-chemical signal (SiA) caused by the labeled analyte (10); and
determining the ratio between the first (Si1) and the analyte signal (SiA), thereby characterizing the analyte (10).

36. The method of claim 34 or 35, wherein an electrode (27) used for the detection is not brought into contact with the first fluid.

37. The method of claim 34 or 35, wherein the analyte (10) is separated from the first fluid and is transferred to a second fluid.

38. The method of claim 34 or 35, wherein the analyte (10) is bound by a catcher molecule (pT, (T)ₙ).

39. The method of claim 38, wherein the catcher molecule (pT, (T)ₙ) is immobilized on a first (16) or second surface.

40. The method of claim 38, wherein the catcher molecule (PT, (T)ₙ) is a nucleic acid, an analogue of a nucleic acid, a peptide nucleic acid (PNA), an antibody, or a receptor.

41. The method of claim 38, wherein the catcher molecule (pT, (T)ₙ) comprises an affinity molecule.

42. The method of claim 34, wherein the first (20) or second probe (22) contains an affinity molecule.

43. The method of claim 35, wherein the first (20) probe or the analyte (10) contains an affinity molecule.

44. The method of claim 42 or 43, wherein the affinity molecule is biotin, avidin, or streptavidin.

45. The method of claim 34 or 35, wherein the analyte (10) is a nucleic acid.

46. The method of claim 45, wherein the nucleic acid has a poly-T-end (pT, (T)ₙ) or a poly-A-end (pA, (A)ₙ).

47. The method of claim 34 or 35, wherein characterizing is determining the length or size of the analyte (10).

48. The method of claim 34 or 35, wherein the analyte (10) is amplified by PCR prior to step a).

49. The method of claim 34 or 35, wherein the analyte (10) is immobilized on a first (16) or second surface.

50. The method of claim 49, wherein the first surface (16) is the surface of a superparamagnetic particle (18).

51. The method of claim 50, wherein the particle (18) has a diameter of 10 nm to 100 μm.

52. The method of claim 50, wherein the particle (18) has a diameter of 1-10 μm.

53. The method of claim 49, wherein the second surface is an electrode (27) used for detection.

54. The method of claim 53, wherein the electrode contains an electrically conductive plastic, an electrically conductive polymer, mercury, amalgam, gold, platinum, carbon, or indium tin oxide.

55. The method of claim 34, wherein the second probe (22) binds specifically to at least one second binding site (14) of the analyte (10).

56. The method of claim 55, wherein the analyte (10) has a known number of first binding sites (12) and an unknown number of second binding sites (14).

57. The method of claim 34 or 35, wherein the analyte (10) is a DNA fragment that exhibits repetitive sequences.

58. The method of claim 57, wherein the second probe binds to the repetitive sequences and the first probe does not bind to the repetitive sequences.

59. The method of claim 57, wherein the repetitive sequences are a consequence of a triplet expansion disease.

60. The method of claim 57, wherein the triplet expansion disease is fragile X syndrome, Huntington's disease, bulbar muscular atrophy, type I spinocerebral ataxia, myotonic dystrophy, or Friedreich's ataxia.

61. The method of claim 49, wherein the first probe (20) is released from the analyte (10) and/or the analyte (10) is released from the first (16) or second surface via heat denaturation, chemical denaturation, enzymatic digestion, or chemical breakdown.

62. The method of claim 34, wherein the first (20) and the second probe (22) are released separately from one another and from the analyte (10) prior to detection.

63. The method of claim 49, wherein the first probe (20) is released from the analyte (10) and the analyte (10) is released from the first (16) or second surface prior to detection.

64. The method of claim 34, wherein the first label (24) and/or the second label (26) are released from the first (20) and/or second probe (22) prior to detection.

65. The method of claim 35, wherein the first label (24) and/or the analyte label (26) are released from the first probe (20) and/or the analyte (10) prior to detection.

66. The method of claim 34 or 35, wherein the first (24) and/or the second label (26) are released by enzymatic digestion or chemical breakdown.

67. The method of claim 34, wherein the first (20) and/or second probe (22) binds sequence-specifically to the analyte (10) via hybridization.

68. The method of claim 34, wherein the first (20) and/or second probe (22) is a nucleic acid, an analogue of a nucleic acid, or a peptide nucleic acid (PNA).

69. The method of claim 34 or 35, wherein the first signal (Si1), second signal (Si2), or analyte signal (SiA) is caused by a catalytic hydrogen release.

70. The method of claim 34 or 35, wherein the labels can be reversibly reduced or oxidized.

71. The method of claim 34 or 35, wherein the labels comprise an osmium complex, a nano gold particle, a cysteine, a ferrocenyle, a daunomycin, a benzoquinone, a naphthoquinone, an anthraquinone or a p-aminophenol group, or a dye.

72. The method of claim 71, wherein the dye is indophenol, thiazine, or phenazine.

73. The method of claim 34, wherein the first (20) or second probe (22) is labeled with multiple labels (24, 26).

74. The method of claim 34, wherein the first (20) or second probe (22) has a linear primary structure on whose one end the label (24, 26) is located.

75. The method of claim 34 or 35, wherein the detection of the first (Si1) and the second signal (Si2) takes place on the same electrode.

76. The method of claim 34 or 35, wherein the detection takes place by means of cathodic stripping voltammetry (CSV), squarewave voltammetry, cyclic voltammetry, or chronopotentiometry.

77. The method of claim 34 or 35, wherein the detection takes place by means of a reversible redox process or a catalytic hydrogen development.
